(19) United States
(12) Patent Application Publication
(10) Pub. No.: US 2022/0142560 A1
(43) Pub. Date: May 12, 2022

(54) MICROBIOME AND METOBOLOME CLUSTERS TO EVALUATE SKIN HEALTH

(71) Applicant: Johnson & Johnson Consumer Inc., Skillman, NJ (US)

(72) Inventors: Thierry Oddos, Clamart (FR); Georgios N. Stamatas, Issy-les-Moulineaux (FR); Pierre-François Roux, Val de Reuil (FR)

(21) Appl. No.: 17/518,151

(22) Filed: Nov. 3, 2021

Related U.S. Application Data
(60) Provisional application No. 63/110,445, filed on Nov. 6, 2020.

(51) Int. Cl. A61B 5/00; C12N 1/20

(52) U.S. Cl. CPC A61B 5/441; C12R 2001/60; C12N 1/205

(57) ABSTRACT

A method for evaluating skin health is disclosed. The method can be used to select skin treatment regimens, ingredients and compositions.

This application claims priority to U.S. provisional patent application No. 63/110,445 the entire contents of which are incorporated by reference herein

### FIELD OF THE INVENTION

The present invention relates to methods for evaluating skin health. The methods may be employed to select skin treatments. The present invention also relates to methods for identifying regimens, ingredients and compositions that can improve the health of skin. It also relates to the use of such regimens, ingredients and compositions to formulate skin care products.

### BACKGROUND OF THE INVENTION

Skin is the body's first line of defense against infections and environmental stressors. It acts as a major physical and immunological protective barrier, but also plays a critical role in temperature regulation, water holding, vitamin D production, and sensing. Its outermost surface consists of a lipid- and protein-laden cornified layer dotted with hair follicles and eccrine glands that secrete lipids, antimicrobial peptides (AMPs), enzymes, salts, etc. It harbors microbial communities living in a range of physiologically and anatomically distinct niches. Overall this constitutes a highly heterogeneous and complex system.

The skin surface is colonized immediately following parturition and is dynamically evolving during the first years of life. While the long-term impact of delivery mode remains unclear, it appears that the skin surface of infants born via cesarean section is predominantly colonized by commensal skin bacteria (Streptococcus, Staphylococcus, Propionibacterium), while the skin surface of vaginally delivered newborns is mostly colonized by microorganisms common to the female urogenital tract (Lactobacillus, Prevotella, Candida)¹⁻⁴. In the first weeks of life, microbial communities start developing site specificity discriminating dry, moist and lipid-rich niches, while increasing in diversity⁵⁻⁷. At puberty, the stimulation of sebaceous gland secretion by hormones markedly shifts the physicochemical properties of the skin surface and favors the development of lipophilic taxa (Corynebacterium and Propionibacterium)⁷. During adulthood though and in the absence of any specific condition, the skin microbiome remains relatively stable⁸, despite the large inter-individual variability⁵, suggesting that mutualistic and commensal interactions exist among microbes and between microbes and host, even for bacterial species often considered as opportunistic pathogens. Under healthy skin conditions, most of the microbes living on the skin behave as commensal or mutualistic organisms. Through various mechanisms, such as the stimulation of innate factor secretion (e.g. IL1α)⁹ or antimicrobial peptides (AMPs), they maintain the microflora composition avoiding the spread of opportunistic parasites¹⁰, while also contributing to the education of the immune system and to healthy skin barrier homeostasis. In case of barrier breach or immunosuppression, these carefully balanced relationships may transition from commensalism to pathogenicity, a transition referred to as dysbiosis¹¹, enabling the overgrowth of pathogenic species, common in skin conditions such as acne¹²⁻¹⁴ psoriasis¹⁵, ulcer¹⁶, and atopic dermatitis¹⁷.

Since the early 1950's, cultured-based studies were undertaken aiming to understand the role of skin microbiome in physiology and disease^{18,19}. The systematic survey of human microbiome has gained significant momentum over the past decade with the advent of 16s RNA profiling and shotgun metagenomic approaches coupled with second generation sequencing technologies. Such methods enable for the identification of potential causal relationships between microbial communities and clinical outcome²⁰. Studies focusing on the role of individual species in skin physiology have followed a reductionistic approach. More recently, the metabolome has emerged as the Rosetta stone warranting the understanding of the molecular bases of microbial influence on host physiology through production, modification, or degradation of bioactive metabolites²¹ in diseases ranging from obesity²², depression²³, autism²⁴, inflammatory bowel disease²⁵, diabetes²⁶, neurological²⁷ as well as heart conditions^{28,29}. Despite being successful in identifying metabolic and bacterial targets to improve health, these more holistic, integrative approaches were so far limited in the study of the gut microbiome.

French Published Application No. 2792728 to L'Oreal discloses a method of evaluating the effects of a product on epidermal lipogenesis that includes applying the product to the surface of a skin equivalent, measuring the variation of a marker of epidermal lipids, then making a comparison with a similar measurement for a control sample.

United States Patent Application No. 20020182112 to Unilever Home & Personal Care USA discloses an in vivo method for measuring the binding of chemical compounds or mixtures of compounds to skin constituents.

United States Patent Application No. 20180185255 to The Procter & Gamble Company discloses a method of selecting a skin cleanser that includes measuring the levels of particular ceramides on the skin both before and after product application and testing for a change in ceramide levels.

U.S. Pat. No. 8,053,003 to Laboratoires Expanscience discloses a method of treating sensitive skin, irritated skin, reactive skin, atopic skin, pruritus, ichtyosis, acne, xerosis, atopic dermatitis, cutaneous desquamation, skin subjected to actinic radiation, or skin subjected to ultraviolet radiation, comprising administering an effective amount of a composition comprising furan lipids of plant oil and thereby increasing synthesis of skin lipids.

U.S. Pat. Nos. 9,808,408 and 10,172,771 to The Procter & Gamble Company discloses a method of identifying a rinse off personal care composition that includes: (a) generating one or more control skin profiles for two or more subjects; (b) contacting at least a portion of skin of the subjects with a rinse-off test composition, rinsing the test composition off the portion of skin, extracting one or more skin samples from each of the subjects, and generating from the extracted samples one or more test profiles for the subjects; (c) comparing the one or more test profiles to the one or more control profiles and identifying the rinse-off test composition as effective for improving the stratum corneum barrier in a human subject who shows (i) a decrease in one or more inflammatory cytokines, (ii) an increase in one or more natural moisturizing factors, (iii) an increase in one or more lipids, and (iv) a decrease in total protein.

Chon et al., Keratinocyte differentiation and upregulation of ceramide synthesis induced by an oat lipid extract via the activation of PPAR pathways, Experimental Dermatology, 24:290-295 (2015), discloses that oat lipids may possess dual agonistic activities for PPARα and PPARβ/δ, increase their gene expression and induce gene differentiation and ceramide synthesis in keratinocytes, which can collectively improve skin barrier function.

Zhang et al., Topically applied ceramide accumulates in skin glyphs, Clinical, Cosmetic and Investigational Dermatology, 8:329-337 (2015), discloses a heterogeneous, sparse spatial distribution of ceramides in stratum corneum.

Ring J. (2016) Pathophysiology of Atopic Dermatitis/Eczema. In: Atopic Dermatitis. Springer, Cham PMID:16098026, discloses the state of the art in research in atopic dermatitis, or atopic eczema.

Glatz et al., Emollient use alters skin barrier and microbes in infants at risk for developing atopic dermatitis, PLoS ONE, 13(2):e0192443 (2018), discloses that emollient use correlated with an increased richness and a trend toward higher bacterial diversity as compared to no emollient use in infants at risk for developing atopic dermatitis.

Capone et al., Effects of emollient use on the developing skin microbiome, presented at the American Academy of Dermatology Annual Meeting, 1-5 Mar. 2019, Washington D.C., USA, discloses that microbial richness is significantly greater with infant wash and lotion than with wash alone. Capone et al. also discloses that both cleansing alone and cleansing and emollient regimens were well tolerated; skin pH remained slightly acidic throughout the study in each regimen; no significant changes for dryness, redness/erythema, rash/irritation, tactile roughness or total score of objective irritation or for overall skin appearance, in either group vs. baseline at any timepoint; an increase in microbial richness seen by 2 and 4 weeks with wash and by 4 weeks with addition of lotion; by 4 weeks use, lotion use increased richness more than wash alone; mild infant wash+lotion routine may best help improve microbial richness, which may contribute to overall skin barrier health by providing the right environment for healthy skin microbes to flourish.

U.S. Pat. No. 9,671,410 and WO2011087523 to The Procter & Gamble Company discloses a screening method for identifying a body wash composition as effective at improving the health of human skin, comprising: a. during a treatment period comprising at least one treatment, contacting a skin surface of a human subject with a body wash composition during a treatment period, wherein the body wash composition is washed off after each application; b. at least once during the treatment period extracting from the epidermis of the human subject (i) at least one biomarker selected from the group consisting of IL Ira and IL1α, (ii) at least one biomarker selected from the group consisting of Trans-Urocanic Acid, Citrulline, Glycine, Histidine, Ornithine, Proline, 2 Pyrrolidone 5 acid, and Serine, (iii) at least one biomarker that is a ceramide, (iv) at least one biomarker that is a fatty acid, and (v) total protein; c. measuring an amount of each biomarker extracted; and d. identifying the body wash composition as effective if the amount of each biomarker is shifted in a direction of improved skin health with total protein decreasing.

U.S. Pat. No. 7,183,057 to Dermtech International discloses a method for detecting a response of a subject to treatment for dermatitis, comprising: a) treating the subject for dermatitis; b) applying an adhesive tape to irritated skin of the subject in a manner sufficient to isolate an epidermal sample, wherein the epidermal sample comprises nucleic acid molecules; and c) detecting expression of a specified gene product, wherein an increase in expression is indicative of response of the subject to treatment for dermatitis, and wherein the method is performed prior to treatment and after treatment.

U.S. Published Application No. 20190136298 to uBiome, Inc. (now Psomagen, Inc.) discloses methods, compositions, and systems for detecting one or more eczema issues by characterizing the microbiome of an individual, monitoring such effects, and/or determining, displaying, or promoting a therapy for the eczema issue.

Co-pending application Ser. No. 16/871,670 discloses in vivo methods for measuring small molecule metabolites in skin. The reference discloses that the methods may be employed to select skin treatments that enhance beneficial metabolite levels in skin.

There remains a need for methods for evaluating skin health.

### SUMMARY OF THE INVENTION

The present invention relates to a method to evaluate skin health.

The invention also relates to a method for screening skin treatment regimens, ingredients and/or compositions, comprising: (a) observing microbiome and metabolome clusters on a surface area of skin prior to application of the skin treatment regimen, ingredient and/or composition; (b) applying the skin treatment regimen, ingredient and/or composition to the area of skin for a period of time; (c) observing microbiome and metabolome clusters on a surface area of said skin after the skin treatment regimen, ingredient and/or composition application on the area of skin; wherein the skin treatment regimen, ingredient and/or composition is beneficial to the skin if the microbiome and metabolome clusters on a surface area of said skin is at least 10% different vs. the no treatment control.

The invention also relates to a method of enhancing skin health, comprising: (a) applying a skin treatment regimen, ingredient and/or composition to skin determined by the screening method above; and (b) repeating (a) for a period of time.

The scope of the present invention will be better understood from the following description.

### BRIEF DESCRIPTION OF THE DRAWINGS

FIG. 1 is a diagram showing the experimental design and analytical strategy employed in the Examples. Skin swabs and tapes were collected from the skin surface of the dorsal forearm of 16 healthy subjects. Each swab sample was subjected to untargeted 16S rRNA sequencing followed by profiling of microbial community taxonomic composition and imputation of functional potential. Each tape sample were analyzed by a combination of UHPLC/MS/MS and GC/MS/MS mass-spectrometry. Parents were asked to fill in a questionnaire to provide information on delivery mode. Skin surface pH and skin surface hydration (SSH) were also recorded on the same individuals on opposite arms.

FIGS. 2A and 2B are barplots depicting the weight of each superpathway (FIG. 2A) and genus (FIG. 2B) in each sample. Areas are color-coded according to super-pathways (metabolome) or phylum (microbiome). The bars on the left show the average distribution across samples. Blacklines delineate individual pathways (FIG. 2A) and genera (FIG. 2B). Overview of the healthy surface skin microbiome and metabolome at the high taxonomic level. Firmicutes are the dominating microbial phyla, while amino acids and lipids are the most prevalent metabolites.

FIGS. 3A to 3D are:
FIG. 3A. Biplot for a factor analysis of mixed data (FAMD). Variables indicated with an outlined triangle are well projected in the reduced dimensional plan (cos 2>0.5).
FIG. 3B. Dotplot depicting the correlation between skin surface hydration (SSH) vs Chao1 alpha diversity index for amplicon sequence variant (ASV).
FIG. 3C. Dotplots depicting the relationship between the Pearson's correlation coefficient between bacterial genus abundance and skin pH, and bacterial genus abundance and SSH. Bacterial genera are color-coded according to the phylum they belong to. More positive correlation to SSH reflects an association between the phylum and a relatively better hydrated environment and the opposite is holds for a negative correlation. More positive correlation to pH reflects an association between the phylum and a relatively alkali environment and the opposite is holds for a negative correlation.
FIG. 3D. Dotplots depicting the relationship between the Pearson's correlation coefficient between metabolic pathways weight and skin pH, and metabolic pathways weight and SSH. Metabolic pathways are color-coded according to the super-pathway they belong to. More positive correlation to SSH reflects an association between the species and a relatively better hydrated environment and the opposite is holds for a negative correlation. More positive correlation to apH reflects an association between the species and a relatively alkali environment and the opposite is holds for a negative correlation.

Skin surface microbiome and metabolome correlate with pH and hydration.

FIGS. 4A to 4C are heatmaps (right) and correlation circles (left) depicting canonical correlations—as defined with regularized generalized canonical correlation analysis—between bacterial phyla and metabolic super-pathways (FIG. 4A), bacterial genus and metabolic pathways (FIG. 4B) and amplicon sequence variants (ASV) and metabolites (FIG. 4C). For (FIG. 4B) and (FIG. 4C), only correlations above R2=0.5 are shown. Skin surface microbiome and metabolome are highly entangled.

1 N-acetylglycine
2 N-acetylasparagine
3 glutamine
4 imidazole propionate
5 anserine
6 N-acetylphenylalanine
7 3-(4-hydroxyphenyl)lactate (HPLA)
8 kynurenine
9 leucine
10 N-acetylleucine
11 4-methyl-2-oxopentanoate
12 3-methyl-2-oxobutyrate
13 methionine sulfoxide
14 urea
15 creatinine
16 4-guanidinobutanoate
17 leucylalanine
18 phenylalanylalanine
19 valylleucine
20 leucylglutamine*
21 phenylacetylglutamine
22 1,5-anhydroglucitol (1.5-AG)
23 lactate
24 ribonate (ribonolactone)
25 arabonate/xylonate
26 mannitol/sorbitol
27 5-dodecenoate (12:1n7)
28 myristate (14:0)
29 pentadecanoate (15:0)
30 palmitate (16:0)
31 margarate (17:0)
32 arachidate (20:0)
33 myristoleate (14:1n5)
34 palmitoleate (16:1n7)
35 hexadecadienoate (16:2n6)
36 (12 or 13)-methylmyristate (a15:0 or i15:0)
37 (14 or 15)-methylpalmitate (a17:0 or i17:0)
38 (16 or 17)-methylstearate (a19:0 or i19:0)
39 glutarate (C5-DC)
40 adipate
41 pimelate (C7-DC)
42 sebacate (C10-DC)
43 tridecanedioate (C13-DC)
44 decanoylcarnitine (C10)
45 2-hydroxypalmitate
46 2-hydroxystearate
47 lianoceroyl ethanolamide (24:0)*
48 glycerol
49 sphinganine
50 N-palmitoyl-sphinganine (d18:0/16:0)
51 N-palmitoyl-sphingosine (d18:1/16:0)
52 N-(2-hydroxypalmitoyl)-sphingosine
(d18:1/16:0(2OH))
53 ceramide (d18:1/17:0, d17:1/18:0)*
54 ceramide (d18:1/20:0, d16:1/22:0,
d20:1/18:0)*
55 cholesterol
56 adenine
57 pseudouridine
58 1-methylnicotinamide
59 N1-Methyl-2-pyridone-5-carboxamide
60 N1-Methyl-4-pyridone-3-carboxamide
61 alpha-tocopherol acetate
62 pyridoxate
63 hippurate
64 benzoate
65 methyl-4-hydroxybenzoate
66 propyl 4-hydroxybenzoate
67 2,3-dihydroxyisovalerate
68 4-acetamidophenol
69 salicylate
70 diglycerol 71 X - 11407 72 X - 12100 73 X -
23737 74 X - 24740 75 X - 24931 76 X - 2564

| | | | |
|---|---|---|---|
| \textcircled{1} | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| \textcircled{2} | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |
| | [symbol] | [symbol] | [symbol] |

[symbol]

indicates data missing or illegible when filed

FIGS. 5A to 5E are:
FIG. 5A. Bi-clustering of metabolome and microbiome data (row Z-score) with the k-means clustering results over-plotted for both individuals and variables and delineating 3 metabolic/microbial clusters.
FIG. 5B. Sample plot from the metabolome perspective.
FIG. 5C. Sample plot from the metabolome perspective.
FIG. 5D. Boxplots showing distribution for pH, skin surface hydration (SSH) and Chao1 microbiome diversity in the three microbial/metabolic clusters.
FIG. 5E. Contingency heatmaps showing the association between the 3 metabolic/microbial clusters and delivery mode.
Unsupervised multi-block sparse partial least square analysis on metabolome and microbiome data unraveled three different surface skin clusters.

FIGS. 6A to 6D are barplots depicting the weight of core metabolites with RA>1.4% in 16 samples (a) of core metabolites with RA>3% in 8 samples (b) top 20 contribution metabolites (c) and core microbial genus with RA>1% in 8 samples (d). The bars on the left of each graph show the average distribution across samples. Overview of the healthy surface skin microbiome and metabolome.

FIGS. 7A to 7C are boxplots highlighting relationships between delivery mode and Chao1 diversity (a), pH (b) and surface skin hydration (SSH, c). FIG. 7D are dotplots depicting correlation between skin surface hydration (SSH, green), pH (red) and Pseudomona, Granulicatella and Cutibacterium abundance. The red and green line correspond to the linear regression for pH (red) and SSH (green). FIG. 7E are dotplots depicting correlation between SSH (green), pH (red) and urea cycle-related metabolites, ceramides and long chain PUFA. The red and green line correspond to the linear regression for pH (red) and SSH (green). Skin surface microbiome and metabolome are highly entangled.

FIGS. 8A and 8B are doplots showing the top correlated metabolites with Cutibacterium relative abundance (RA, a) and Staphyloccocus RA (b). Top correlated metabolites from the lipid category for Cutibacterium and from the amino acid category for Staphylococcus.

### DETAILED DESCRIPTION OF THE INVENTION

While the infant skin metabolome is dominated by amino acids, lipids and xenobiotics, the primary phyla of the microbiome are Firmicutes, Actinobacteria and Proteobacteria. Zooming in to the species level revealed a large contribution of commensals belonging to Cutibacterium and Staphylococcus genera, including Cutibacterium acnes, Staphylococcus epidermidis, Staphylococcus aureus and Staphylococcus hominis. This heterogeneity is further reflected when combining the microbiome with metabolome data. Integrative analyses enabled the present inventors to delineate the co-existence of three distinct metabolic/microbial clusters at the skin surface of infants: a) one built on the association between Cutibacterium, Actinomyces and Bergeyella favored by a ceramide- and lipid-rich, relatively dryer and more basic environment, b) one consisting of the association of multiple commensals such as Corynebacterium, Lactobacillus, Clostridium, Escherichia, Pseudomonas and Staphylococcus in a lysine- and sugar-rich, relatively more hydrated and acidic environment, c) one dominated by Streptococcus that is independent of the presence of any particular metabolomic profile.

The discovery of the presence of microbe/metabolite functional clusters is an important step in understanding the host-microbiome interaction and how it affects skin health. Specifically, the cluster dominated by Cutibacterium appears to be linked to the formation of the hydrophobic skin barrier, while the cluster associated with amino acids appears to be relevant to the water holding capacity and pH regulation of the skin surface. Such important insights open new areas of research for more refined questions regarding the mechanistic understanding of the microbiome role in the skin's physiological function.

### Definitions

As used herein, the following terms shall have the meaning specified thereafter:

A “barplot” a graphic that shows the relationship between a numeric and a categoric variable. Each entity of the categoric variable is represented as a bar. The size of the bar represents its numeric value.

“Bi-clustering” is a data mining technique that allows simultaneous clustering of the rows and columns of a matrix that is used to study gene expression data, especially for discovering functionally related gene sets under different subsets of experimental conditions.

A “biplot” is plot which represents both the observations and variables of a matrix of multivariate data on the same plot.

“Ceramides” as used herein refers to a family of lipid molecules that makeup part of the stratum corneum layer of the skin. Together with cholesterol and saturated fatty acids, ceramides help the skin to be water-impermeable to help prevent water loss and also to act as a protective layer to keep unwanted microorganisms from entering the body through the skin. When the ceramide level of skin is suboptimal, the stratum corneum can become compromised. The skin can also become dry and irritated. Ceramides are composed of a fatty acid chain amide linked to a sphingoid base. There are three types of fatty acids which can be part of a ceramide. These are non-hydroxy fatty acids (N), α-hydroxy fatty acids (A), and esterified S2-hydroxy fatty acids (EO). In addition, there are four sphingoid bases: dihydrosphingosine (DS), sphingosine (S), phytosphingosine (P), and 6-hydroxy sphingosine (H).

“Comprising” as used herein is inclusive and does not exclude additional, unrecited elements, steps or methods. Terms as used herein that are synonymous with “comprising” include “including,” “containing,” and “characterized by,” and mean that other steps and other ingredients can be included. The term “comprising” encompasses the terms “consisting of” and “consisting essentially of,” wherein these latter terms are exclusive and are limited in that additional, unrecited elements, steps or methods ingredients may be excluded. The skin treatment regimens, ingredients and compositions of the present disclosure can comprise, consist of, or consist essentially of, the steps, methods and elements as described herein.

A “dotplot” is a type of graphic display used to compare frequency counts within categories or groups made up of dots plotted on a graph.

“Effective amount” as used herein means an amount of a regimen, ingredient and/or composition sufficient to significantly induce a positive skin benefit, including independently or in combination with other benefits disclosed herein. This means that the content and/or concentration of active component in the regimen, ingredient and/or composition is sufficient that when the regimen, ingredient and/or composition is applied with normal frequency and in a normal amount, the regimen, ingredient and/or composition can result in the treatment of one or more undesired skin conditions. For instance, the amount can be an amount sufficient to inhibit or enhance some biochemical function occurring within the skin. This amount of active component may vary depending upon, among other factors, the type of regimen, ingredient and/or composition and the type of skin condition to be addressed.

“Epidermis” as used herein refers to the outer layer of skin, and is divided into five strata, which include the: stratum corneum, stratum lucidum, stratum granulosum, stratum spinosum, and stratum basale. The stratum corneum contains many layers of dead, a nucleated keratinocytes that are essentially filled with keratin. The outermost layers of the stratum corneum are constantly shed, even in healthy skin. The stratum lucidum contains two to three layers of a nucleated cells. The stratum granulosum contains two to four layers of cells that are held together by desmosomes that contain keratohyaline granules. The stratum spinosum contains eight to ten layers of modestly active dividing cells that are also held together by desmosomes. The stratum basale contains a single layer of columnar cells that actively divide by mitosis and provide the cells that are destined to migrate through the upper epidermal layers to the stratum corneum. The predominant cell type of the epidermis is the keratinocyte. These cells are formed in the basal layer and exist through the epidermal strata to the granular layer at which they transform into the cells know as corneocytes or squames that form the stratum corneum. During this transformation process, the nucleus is digested, the cytoplasm disappears, the lipids are released into the intercellular space, keratin intermediate filaments aggregate to form microfibrils, and the cell membrane is replaced by a cell envelope made of cross-linked protein with lipids covalently attached to its surface. Keratins are the major structural proteins of the stratum corneum. Corneocytes regularly slough off (a process known as desquamation) to complete an overall process that takes about a month in healthy human skin. In stratum corneum that is desquamating at its normal rate, corneocytes persist in the stratum corneum for approximately 2 weeks before being shed into the environment.

“Epithelial tissue” as used herein refers to all or any portion of the epithelia, in particular the epidermis, and includes one or more portions of epithelia that may be obtained from a subject by a harvesting technique known in the art, including those described herein. By way of example and without being limiting, epithelial tissue refers to cellular fragments and debris, proteins, isolated cells from the epithelia including harvested and cultured cells.

“Metabolite” as used herein refers to the intermediate end product of metabolism. The term metabolite is usually restricted to small molecules. Metabolites have various functions, including fuel, structure, signaling, stimulatory and inhibitory effects on enzymes, catalytic activity of their own (usually as a cofactor to an enzyme), defense, and interactions with other organisms (e.g. pigments, odorants, and pheromones). A primary metabolite is directly involved in normal “growth”, development, and reproduction. A secondary metabolite is not directly involved in those processes, but usually has an important ecological function.

“Metabolomics” as used herein refers to the study of the small-molecule metabolite profile of a biological organism, with the metabolome jointly representing all metabolites. The “metabolome” is the very end product of the genetic setup of an organism, as well as the sum of all influences it is exposed to, such as nutrition, environmental factors, and/or treatment.

“Microbiome” as used herein refers to a characteristic microbial community occupying a reasonable well-defined habitat which has distinct physio-chemical properties. The microbiome not only refers to the microorganisms involved but also encompass their theatre of activity, which results in the formation of specific ecological niches. The microbiome, which forms a dynamic and interactive micro-ecosystem prone to change in time and scale, is integrated in macro-ecosystems including eukaryotic hosts, and here crucial for their functioning and health.¹ ¹ Berg, G., Rybakova, D., Fischer, D. et al. Microbiome definition re-visited: old concepts and new challenges. Microbiome 8, 103 (2020). https://doi.org/10.1186/s40168-020-00875-0.

“Microbiota” consists of the assembly of microorganisms belonging to different kingdoms (Prokaryotes [Bacteria, Archaea], Eukaryotes [e.g., Protozoa, Fungi, and Algae]), while “their theatre of activity” includes microbial structures, metabolites, mobile genetic elements (e.g., transposons, phages, and viruses), and relic DNA embedded in the environmental conditions of the habitat.² ² Id.

“Skin” is divided into three main structural layers, the outer epidermis, the inner dermis, and the subcutaneous tissue.

“stratum corneum” as used herein, refers to the outermost layer of the epithelia, or the epidermis, and is the skin structure that provides a chemical and physical barrier between the body of an animal and the environment. The stratum corneum is a densely packed structure comprising an intracellular fibrous matrix that is hydrophilic and able to trap and retain water. The intercellular space is filled with lipids formed and secreted by keratinocytes and which provide a diffusion pathway to channel substances with low solubility in water.

“Subject” as used herein refers to a human for whom a regimen, ingredient and/or composition is tested or on whom a regimen, ingredient and/or composition is used in accordance with the methods described herein.

“Substantially free of” as used herein, unless otherwise specified, means that the regimen, ingredient and/or composition comprises less than about 2%, less than about 1%, less than about 0.5%, or even less than about 0.1% of the stated ingredient. The term “free of”, as used herein, means that the regimen, ingredient and/or composition comprises 0% of the stated ingredient. However, these ingredients may incidentally form as a by-product or a reaction product of the other components of the regimen, ingredient and/or composition.

“Test ingredients and/or compositions” as used herein include and encompass purified or substantially pure ingredients and/or compositions, as well as formulations comprising one or multiple ingredients and/or compositions. Thus, non-limiting examples of test ingredients and/or compositions include water, a pharmaceutical or cosmeceutical, a product, a mixture of compounds or products, and other examples and combinations and dilutions thereof.

“Test surfaces” as used herein means a region of epithelia tissue which has been contacted with and/or by a product, such as a consumer product and/or a test regimen, ingredient and/or composition, whereby the contact of the product and/or the regimen, ingredient and/or composition on the epithelia tissue has resulted in some change, such as but not limited to, physiological, biochemical, visible, and/or tactile changes, in and/or on the epithelia tissue that may be positive or negative. In some examples, positive effects caused by regimen, ingredient and/or composition may include but are not limited to, reduction in one or more of erythema, trans-epidermal water loss (TEWL), discoloration of the skin, rash, dermatitis, inflammation, eczema, dandruff, edema and the like. The location of the affected surface will depend upon the regimen, ingredient and/or composition used or the location of some physiological, biochemical, visible, and/or tactile change in and/or on the epithelia tissue.

“Topical application”, “topically”, and “topical”, as used herein, mean to apply the regimen, ingredient and/or composition used in accordance with the present disclosure onto the surface of the skin.

“Treating” or “treatment” or “treat” as used herein includes regulating and/or immediately improving skin appearance and/or feel.

A skin treatment regimen, ingredient and/or composition can be formulated to not only minimize any negative impact on skin, but to enhance the stratum corneum for enhanced skin barrier function and hydration. This also allows for such skin treatment regimen, ingredient and/or composition to be screened for skin mildness and barrier improvement. This could be done, for example, by having subjects use the skin treatment regimen, ingredient and/or composition and measuring the impact on microbiome and metabolome clusters.

Shifts due to skin treatments in the relative abundance/presence/influence of the microbiome/metabolome clusters can be observed and treatment benefits on skin moisturization and skin barrier function can be deduced. The presence of xenobiotics (that include left over residues of previous skincare treatments and other environmental exposures) and their influence on the clusters and on skin health can also be observed.

Additional optional materials can also be added to the composition to treat the skin, or to modify the aesthetics of the composition as is the case with perfumes, colorants, dyes, or the like.

Other optional materials can be those materials approved for use in cosmetics and that are described in the International Cosmetic Ingredient Dictionary and Handbook, Sixteenth Edition, Personal Care Products Council, 2016.

U.S. Pat. No. 10,267,777 to Metabolon, Inc. discloses a mass spectrometry method of measuring levels of small molecules in a sample from an individual subject to determine small molecules having aberrant levels in the sample from the individual subject, the determination being relevant to screening for a plurality of diseases or disorders in the individual subject or relevant to facilitating diagnosis of a plurality of diseases or disorders in the individual subject.

U.S. Pat. No. 8,849,577 to Metabolon, Inc. discloses a method for identifying biochemical pathways affected by an agent comprising: obtaining a small molecule profile of a sample from an assay treated with said agent, said small molecule profile comprising information regarding at least ten small molecules including identification information for the at least ten small molecules; comparing said small molecule profile to a standard small molecule profile; identifying components of said small molecule profile affected by said agent; identifying one or more biochemical pathways associated with said identified components by mapping said identified components to the one or more biochemical pathways using a collection of data describing a plurality of biochemical pathways and an analysis facility executing on a processor of a computing device, thus identifying biochemical pathways affected by said agent, wherein the plurality of biochemical pathways includes the one or more identified biochemical pathways associated with the identified components and a plurality of non-identified biochemical pathways; and storing information regarding each identified biochemical pathway and an identified component or identified components mapped to the identified biochemical pathway for each identified biochemical pathway.

U.S. Published Application No. 20160356798 to Metabolon, Inc. discloses a method of estimating de novo lipogenesis in a subject.

U.S. Published Application No. 20160019335 to Metabolon, Inc. discloses a method for analyzing metabolite data in a sample.

U.S. Published Application No. 20140287936 to Metabolon, Inc. discloses a method for identifying small molecules relevant to a disease state.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### Examples

The following examples describe and demonstrate examples within the scope of the invention. The examples are given solely for the purpose of illustration and are not to be construed as limitations of the present invention, as many variations thereof are possible without departing from the spirit and scope of the invention.

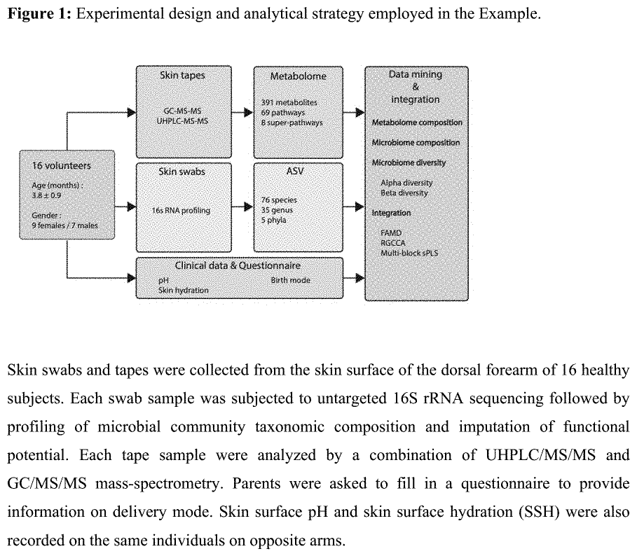

To characterize the skin metabolic profile and microbiome composition, dorsal forearm skin tapes and swabs from a cohort including 16 healthy subjects (9 females, 7 males, 118±29 days old in average were collected and analyzed, FIG. 1 and Table S1, see Methods section for an overview of inclusion and exclusion criteria). In addition, parents were asked to fill in a questionnaire to provide information on delivery mode. Surface skin pH and surface skin hydration (SSH) values were also recorded. Matched swab samples (left and right arms) were subjected to untargeted 16S rRNA sequencing followed by profiling of microbial community taxonomic composition defining amplicon sequence variants (ASV). Skin tapes were analyzed by a combination of UHPLC/MS/MS and GC/MS/MS. The profiling was carried-out using sensitive, high-resolution mass-spectrometers in non-targeted mode, capturing a large number of known and uncharacterized metabolites.

### Overview of the Healthy Skin Surface Microbiome and Metabolome

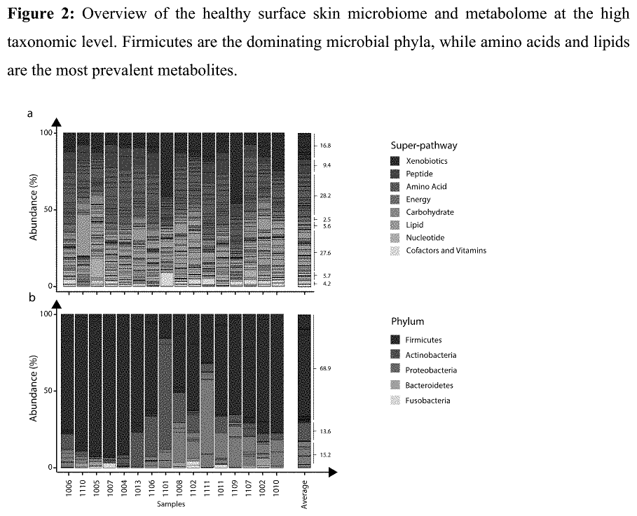

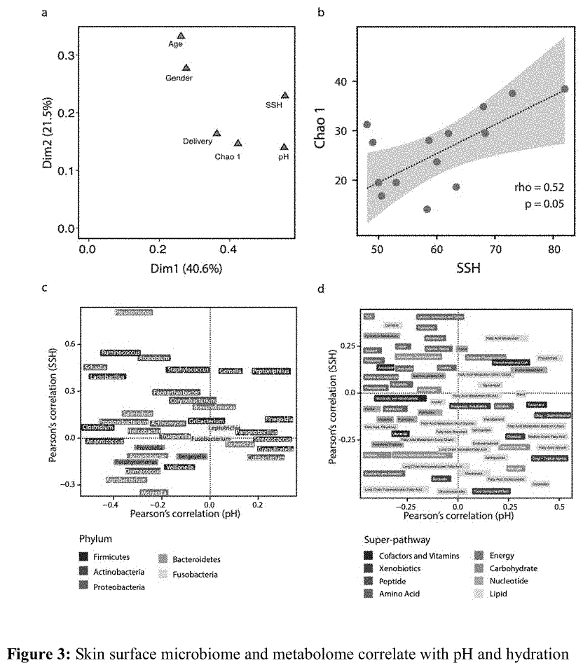

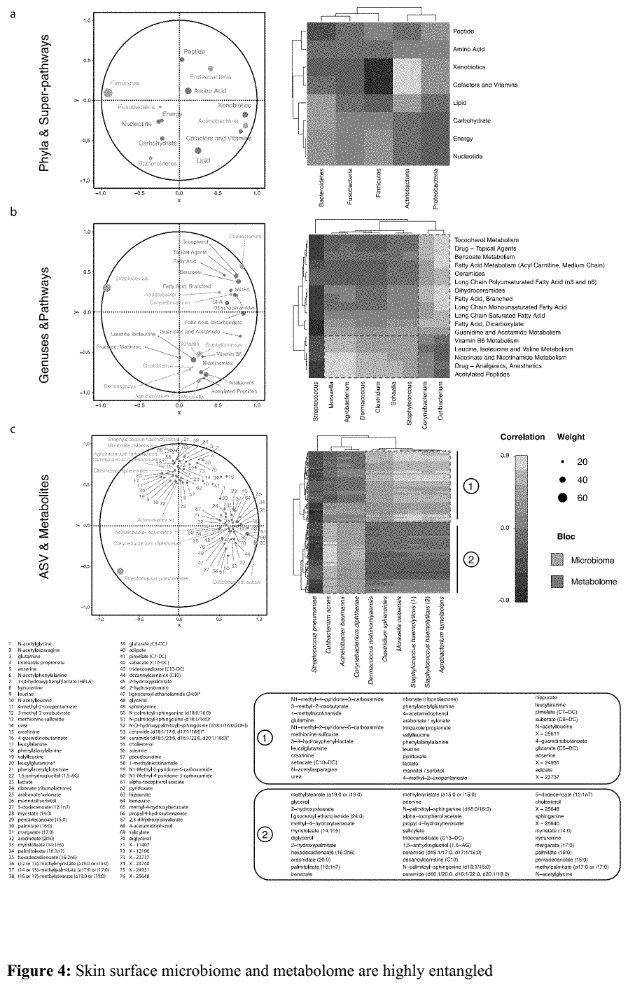

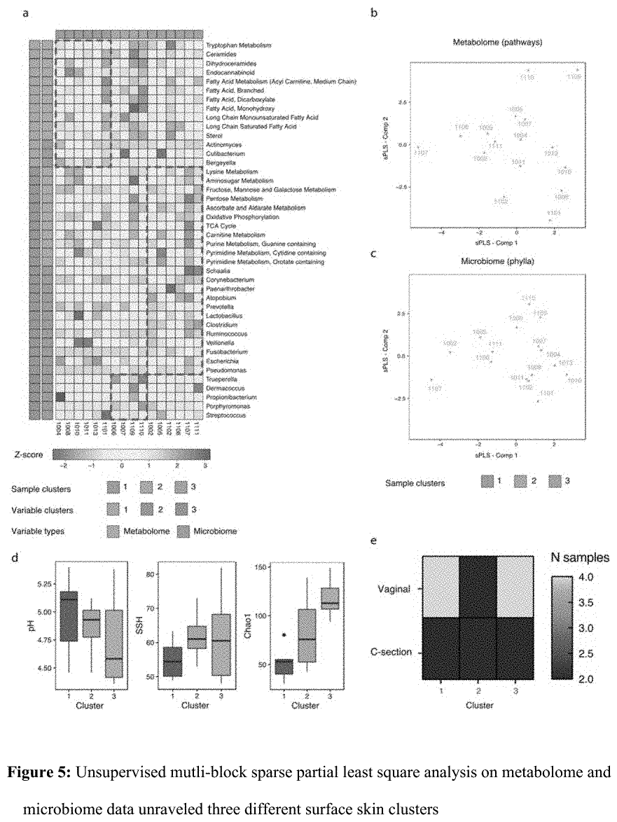

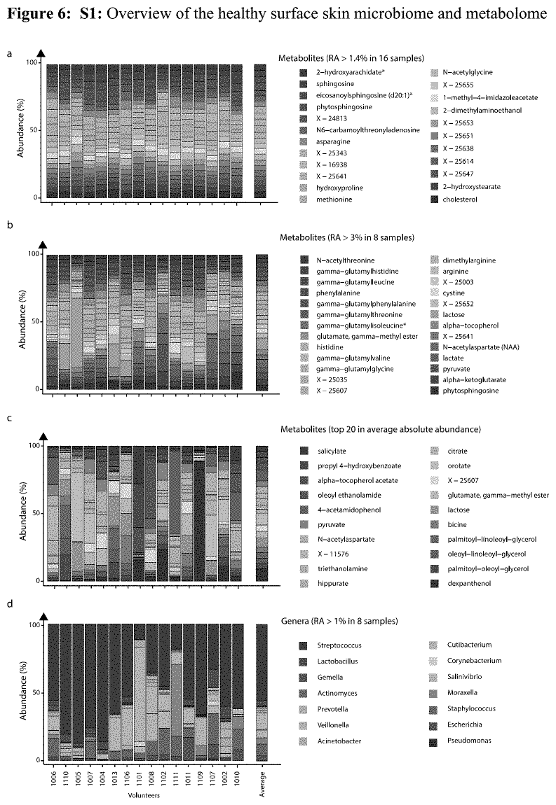

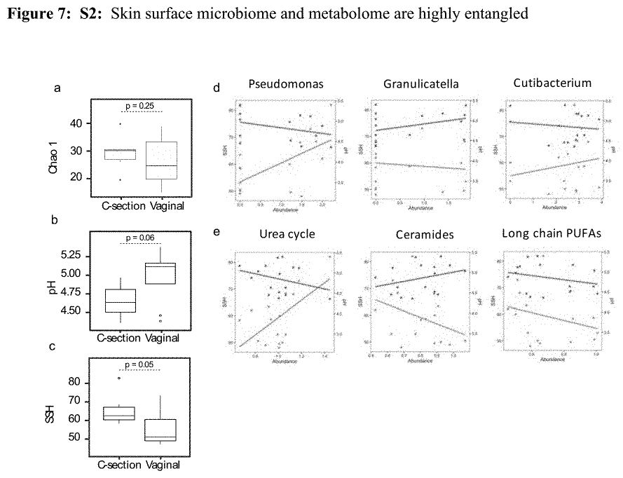

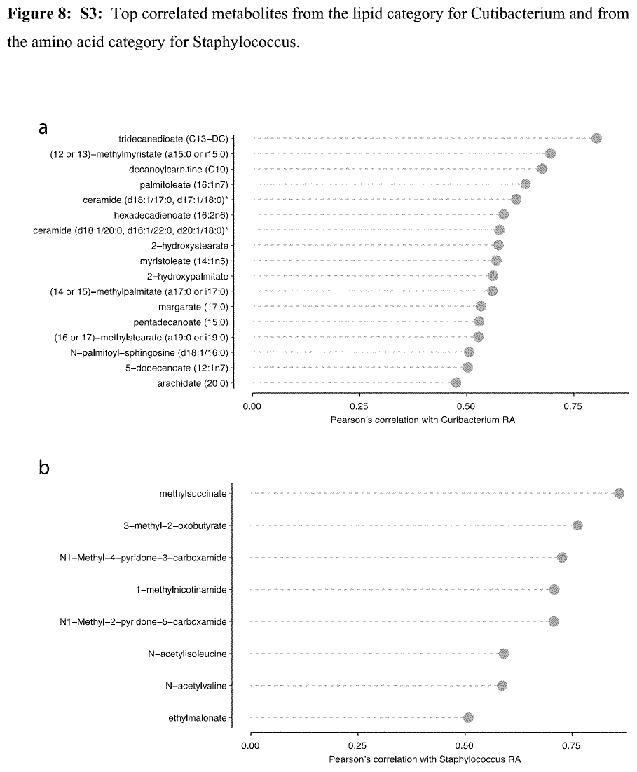

The composition and heterogeneity of the skin microbiome and metabolome in this cohort were analyzed, first by estimating the relative contribution of each metabolic pathway and bacterial taxum, grouped into super-pathways and phyla respectively. Overall, from the metabolome perspective, the leading super-pathways are amino acids (28.2% of total metabolites), lipids (17.6%) and xenobiotics (16.8%), and from the microbiome perspective, the leading phyla are Firmicutes (68.9%), Proteobacteria (15.2%) and Actinobacteria (13.6%) (FIG. 2). Table S2 contains raw metabolomic data and Table S3 contains raw microbiome data.

The core metabolome, which consists of 24 metabolites present in all the samples at 1.4% relative abundance, contains fatty acid derivatives (2-hysroxyarachidate, eicosanoylsphingosine, phytosphingosine), amino acid and derivatives (asparagine, hydroxyproline, methionine, N-acetylglycine, dimethylaminoethanol), nucleosides (N6-carbamoylthreonyladenosine), carboxylic acids (1-methyl-4-imidazoleacetic acid) as well as uncharacterized compounds, in even proportion across all subjects (FIG. 6 (S1A)). Lowering the prevalence threshold to 8 samples while increasing the abundance threshold to 3% revealed that amino-acids (N-acethyltrheonine, phenylalanine, arginine, histidine, gamma-gluthamylhistindine, gamma-glutamylleucine, etc.) were largely contributing to the core metabolome, together with Kreb's cycle and (an)aerobic cellular respiration by-products (alpha-ketoglutarate, pyruvate, lactate), alpha-tocopherol and lactose (and FIG. 6 (S1B)). When focusing only on metabolites that are in average contributing the most to the overall skin metabolome without putting any restriction in term of prevalence, it was found that among the most abundant compounds, a significant proportion belong to the xenobiotics group (salicylate, propyl 4-hydroxybenzoate, 4-acetamidophenol, triethanolamine, bicine, dexpanthenol) likely originating from skincare routine (FIG. 6 (SIC)).

The core skin microbiome, which consists of 14 genera present in at least 8 samples at 1% relative abundance, is largely dominated by Streptoccocus (52.8%), Cutibacterium (11.8%) and Staphylococcus (8.1%) (FIG. 6 (SID)). This overall contribution of major genera is highly heterogenous across samples: for example, the microbiome from sample 1101 is dominated by Cutibacterium (≈75% of the core microbiome), while the one from sample 1111 is leaded by Moraxella (≈50% of the core microbiome).

### The Skin Surface Metabolome Shapes Bacterial Communities and Impacts Microbiome Diversity

To visualize the relationship between clinical data, individual skin physico-chemical properties and microbial richness, factor analysis of mixed data (FAMD), a principal component method dedicated to exploring data with both continuous and categorical variables, was employed (FIG. 3A). This analysis revealed an overall association between skin pH, microbiome diversity (Chao1) and SSH. Looking at individual pairwise correlations, a positive correlation between SSH and microbial richness was confirmed (FIG. 3B). Interestingly, while the birth mode appears to influence skin surface pH and SSH values, no influence on skin microbial diversity was detected on this cohort of infants 3-6 months after birth (FIG. 7 (S2A, S2B and S2E)).

To explore the association between the skin micro-environment of the individual (skin pH and SSH) and bacterial communities, the pairwise Pearson's correlation coefficient skin between skin pH and bacterial genera abundance, and between SSH and bacterial genera abundance was computed. By combining in a single graph the coefficient values of the two correlations (genera abundance-pH vs genera abundance-SHH), the affinity of each genus for distinct skin niches in terms of acidity and moisturization could be determined (FIG. 3C, FIG. 7 (S2D)). While Pseudomonas, Ruminococcus, Atopobium, Schaalia, and Lactobacillus favor individuals with relatively more acidic and hydrated skin, Cutibacterium is found in individuals with relatively more basic and slightly drier skin, and Moraxella, Agrobacterium and Acinetobacter in those with slightly acid and slightly dry skin. This analysis also revealed that the genera inside a given phylum were settling in heterogeneous niches, hence the significance to study microbiome at the finest possible grain.

We then performed the same analysis focusing on metabolites (FIG. 3D, FIG. 7 (S2E)). As expected, amino-acids and TCA- and urea-cycle derived metabolites were mostly associated with individuals with more acidic and more hydrated skin. A broad distribution of lipid-related metabolites across niches, reflecting the broad spectra of chemical properties of these metabolite class, was observed. Indeed, while long chain unsaturated fatty acids tend to associate with individuals with slightly more acidic and drier skin, phospholipids are in higher proportion at relatively more basic and more hydrated sites and ceramides enriched in relatively more basic and drier niches.

Skin Microbiome Aggregates Around 3 Distinct Communities Characterized by their Metabolic Microenvironment

To resolve the complex relationships connecting microbiome and metabolome, a regularized Canonical Correlation Analysis (rCCA) integrating both microbiome and metabolome at different taxonomic levels: 1) bacterial phyla vs metabolic superpathways, 2) bacterial genera vs metabolic pathways, and 3) bacterial species vs metabolites was applied. At the higher taxonomic level, this analysis reveals a strong positive correlation between the abundance of xenobiotics, cofactors and vitamins and the relative abundance of Actinobacteria, as well as a strong anti-correlation between the aforementioned metabolic superpathways and Firmicutes (FIG. 4A). Zooming-in at the genus and metabolic pathways levels revealed three major clusters: a) the first one built on the association between Cutibacterium, Acinetobacter and Corynebacterium in a niche enriched in fatty acid (free-, mono-unsaturated-, saturated fatty acids), benzoate, tocopherol and dihydroceramides (FIG. 8 (S3A)), b) the second one associating Dermacoccus, Agrobacterium, Moraxella, Schaalia, Clostridium and Staphylococcus with sugars (fructose, manose), amino acids (leucine, isoleucine), peptides and vitamin B6 (FIG. 8 (S3B)), and c) the last one dominated by Streptococcus in an niche independent of any particular correlation with the aforementioned metabolic pathways (FIG. 4B). The composition of these three communities can be characterized in more detail, when the microbiome and metabolome data at the species and individual metabolite levels are examined (FIG. 4C).

To validate this observation, multi-omic sparse Partial Least Square unsupervised analysis, integrating microbiome genera abundance data together with metabolome abundance data was applied (FIG. 5A). Retaining 15 variables in each ‘omic bloc was sufficient to properly discriminate three clusters of metabolomic and microbe variables splitting the samples in three different groups (FIGS. 5B and 5C). The first group of samples (violet cluster) is characterized by an association between fatty-acid metabolites, ceramides with Cutibacterium, Actinobacterium and Bergeyella and is less rich from the microbiome perspective (FIG. 5D). The second group of samples (turquoise cluster) is driven by the association between Streptococcus, Porphyroimona, Propionibacterium, Dermacoccus and Trueperella in a niche mostly independent of the presence of fatty acids, ceramides, sugars and pyrimidine, and is richer from the microbiome perspective (FIG. 5D). The third group (green) is built on top of a richer microbiome associating Schaalia, Corynebacterium, Atopobium, Lactobacillus, Clostridium, Escherischia growing in an environment rich in lysine, sugar, TCA. Overall, children born vaginally tend to host more frequently the cluster one and three (FIG. 5E).

### Discussion

Since the late 19^{th} century the presence of microbes has been associated with disease. However, mostly through a better understanding of the GI system, we have come to realize that there are commensal and mutualistic species living inside and on us. The particular anatomic location and function of skin as the interface between the organism and the environment, where microbes are ubiquitous, makes it suitable for microbial colonization. We now understand the skin microbiome as an integral part of the organism interface with the environment, which among others, restrains potential colonization by opportunistic pathogens. However, the actual mechanisms of microbe-host interactions and the role of the microbiome in skin physiology remain obscure.

As it is the case for the whole human organism, skin is undergoing dramatic changes after birth. At parturition, the newborn starts its journey shifting from a constant-temperature, wet and sheltered environment to a dry highly variable surrounding, potentiating water-loss, mechanical trauma and infections. Despite the fact that its development starts early during the first pregnancy trimester in utero, in preparation for the later development of a functional stratum corneum (SC)^{31,32}, neonatal skin is still immature at birth relative to adult and gradually follows a maturation process during the first years of life³³⁻³⁵. It is now established that SC is thinner^{33,36} and dryer³⁶⁻⁴⁰, corneocytes are smaller³³, collagen fibers less dense³³, and that skin contains overall less natural moisturizing factor (NMF)³⁴ and lipids in infants compared to adults. These factors directly impact the skin barrier properties and physico-chemical conditions at the skin surface.

Exploiting the skin microbiome to treat skin conditions and to develop innovative topical treatments requires a detailed knowledge of the crosstalk connecting the microbial community to host physiology, which is currently missing. To fill this critical gap in our knowledge, the present inventors used a multidimensional approach at high resolution combining 16sRNA sequencing and untargeted metabolomics in samples taken from healthy infant skin surface. State-of-the art dimension reduction methodologies was further applied to better understand how the microbiome shapes and is being shaped by the skin micro-environment in healthy conditions.

Despite a relatively homogeneous distribution of the major phyla and the metabolic super-pathways, a more granular analysis of these two components revealed a substantial heterogeneity between samples. While amino acids, lipids and xenobiotics were dominating together with Firmicutes, Actinobacteria and Proteobacteria as already shown in neonates⁴, zooming in to lower taxonomic levels revealed a large contribution of commensals belonging to the Cutibacterium and Staphylococcus genera, including species such as Cutibacterium acnes, Staphylococcus epidermidis, Staphylococcus aureus, Staphylococcus hominis or Streptococcus pneumoniae. As reported in other works the present inventors found that even in healthy skin species commonly driving dysbiosis^{10,11,17} exist.

This heterogeneity is further reflected in the association between the microbiome and the metabolome at the skin surface. Integrative analyses indeed enabled the present inventors to delineate the existence of three distinct metabolic/microbial clusters at the skin surface in infants: a) one build on the association between Cutibacterium, Actinomyces and Bergeyella in individuals with ceramide- and lipid-rich, relatively drier and basic skin surface, b) one consisting of the association of multiple commensals such as Corynebacterium, Lactobacillus, Clostridium, Escherichia, Pseudomonas and Staphylococcus in individuals with a lysine- and sugar-rich, relatively moistened and more acidic skin surface, c) one that is anticorrelated or independent of a particular metabolite microenvironment.

Cutibacterium acnes is a major skin commensal, and is the dominating species of the pilosebaceous gland, accounting for up to 90% of the total microbiome in sebum rich sites such as the scalp or the face⁶. While accumulating evidence shows its role in enhancing sebaceous gland lipogenesis and triglycerides synthesis in vitro and in vivo⁴¹, its interplay with stratum corneum lipid metabolism remains elusive. The data herein highlights that C. acnes has a greater affinity for lipid-rich skin surface and accumulates at sites with greater amounts of fatty acids (2-hydroxystearate, 2-hydroxypalmitate, myristoleate, arachidate, palmitoleate), cholesterol and ceramides (N-palmitoyl-sphinganine, N-palmitoyl-sphingosine, N-2-hydroxypalmitoyl-sphingosine, N-stearoyl-D-sphingosine, N-arachidoyl-D-sphingosine). Whether organized into broad bilayers in the inter-corneocyte spaces, or covalently bound to the corneocyte envelope in the stratum corneum, lipids are essential constituents of the human epidermis, supporting skin barrier function, cell signaling and anti-microbial defense⁴². Considering both lipid functional implications in epidermis physiology and C. acnes implication in acne vulgaris pathogenesis, these results are of utmost relevance.

Staphylococcus aureus is known to be involved in the pathology of atopic dermatitis (Leyden J J, Marples R R, Kligman A M. 1974. Staphylococcus aureus in the lesions of atopic dermatitis. Br J Dermatol 90: 525-530). In fact, the relative abundance of S. aureus dominates the microbiome composition on atopic lesions and is responsible for the observed decline in the overall microbiome diversity (Kong H H et al. Genome Res 2012 22(5):850-9). This species relies on the branched-chain amino acids (isoleucine, leucine, valine) for the synthesis of proteins and membrane branched-chain fatty acids. These amino-acids are therefore crucial for its metabolism, adaptation and virulence⁴³.

### Methods

### Clinical Study, Measurements and Sample Collection

A single-center, randomized, evaluator-blind, 5-week trial (NCT03457857) was conducted to assess the effects of two skincare regimens on the cutaneous microbiome, metabolome, and skin physiology of healthy infants aged between 3-6 months in general good health based on medical history and without any skin conditions or family history of known allergies. Baseline data was used to assess the crosstalk between microbiome, metabolome and skin physiology. An institutional review board (IRB; IntegReview, Austin, Tex.) approved the study and parents/legally authorized representatives (LARs) of study participants provided written informed consent. Parents/LARs of prospective participants were screened for eligibility criteria using an IRB approved screener. Parents/LARs were required to be at least 18 years of age. Participant eligibility was assessed at an initial screening visit by the primary investigator, and the study physician confirmed eligibility of each participant before enrollment. All eligible study participants entered a 7-day washout period, during which parents/LARs were instructed to use a marketed gentle baby cleanser (JOHNSON'S® HEAD-TO-TOE® Wash & Shampoo: Johnson & Johnson Consumer Inc., Skillman, N.J., USA) in place of their infant's normal body cleanser, at least 3 times during the week, and to refrain from use of any type of moisturizer or lotion. Sample collection from left or right dorsal forearm was determined by randomization, with one arm used for skin swabs for microbiome analysis and skin tape samples for metabolomic analysis, and the opposite arm used for skin surface hydration (SSH) and skin pH readings. SSH was assessed using a Corneometer CM825 (Courage-Khazaka Electronic GmbH, Cologne, Germany), using 3 consecutive readings from the subject's dorsal forearm.

Skin pH measurements were obtained from 5 consecutive readings within each test site on the subject's dorsal forearm, using a Skin-pH-Meter® (PH 905, Courage and Khazaka, Cologne, Germany). Skin swab samples were sent to an independent laboratory (RTL Genomics, Lubbock, Tex., USA) for DNA extraction and sequencing of the skin microflora. Sequencing was performing using primers targeting the 16S regions. Two consecutive skin tape samples were collected from the dorsal forearm, adjacent to the site used for microbial sample collection. Samples were collected using D-Squame Standard Sampling Discs (CuDerm Corporation, Dallas, Tex., USA) with 30 seconds of constant pressure. The tape was then removed with forceps and placed into a scintillation vial (adhesive side in) and immediately stored at −80° C. Metabolomic analysis was performed by an independent laboratory (Metabolon, Morrisville, N.C., USA).

### Microbiome Profiling

To profile skin microbiota, sequencing was conducted by RTLGenomics (Lubbock, Tex., USA). Briefly, DNA was extracted using Qiagen's MagAttract PowerSoil DNA Isolation on the Thermo Kingfisher 96-well extraction robot following manufacturer's instructions. Sample amplification for sequencing was conducted using primers encompassing variable regions 1 through 3 of the 16s rRNA gene as previously described⁴⁴. Sequencing was conducted on the Illumina MiSeq platform (Illumina, San Diego, Calif.) using manufacture protocol and targeting a minimum depth of 10,000 taxonomically classified reads per sample. Raw paired-end sequencing reads were first merged using custom R script and PCR primers were removed from the obtained sequences. These sequences were further quality-trimmed, filtered and denoised using DADA2 framework⁴⁵ to infer amplicon sequence variants (ASV). Among the 1647259 read pairs generated, 1071553 were kept. Taxonomy was assigned using the HiMAP NCBI-derived database⁴⁶. ASV abundance matrix, sample metainformation and taxonomy were finally stored as a phyloseq object⁴⁷. ASV detected in less than two samples were excluded from the analysis.

### Metabolomics

Untargeted metabolomics profiling of the skin samples was performed by Metabolon, Inc. (Durham, N.C., USA) as previously described⁴⁸. Compounds were identified by comparison to library entries of purified standards or recurrent unknown entities. Metabolon maintains a library based on more than 4500 authenticated purified standards that contains the retention time/index (RI), mass to charge ratio (m/z), and chromatographic data (including MS/MS spectral data) on all molecules present in the library. The peak intensities corresponding to each metabolite were normalized to the total intensity count for a given sample.

### Statistical Analyses

The analyses were performed in R v4.0.0 and rely on the packages mixOmics⁴⁹, FactorMineR⁵⁰, vegan, and phyloseq⁴⁷. Factorial Analysis of Mixed Data (FAMD) was applied on a matrix containing pH, SSH, microbiome Chao1 index, as well as gender and mode of birth information for each sample. Regularized Canonical Correlation Analysis (rCCA) was performed on the combination of the metabolomic abundance matrix and the microbiome relative abundance matrix after regularization through Ridge regression (ι2 penalties) of parameters λ1 and λ2 using a leave-one-out cross-validation procedure. To define metabolic/microbial clusters, a block sparse Partial Least Square (PLS) analysis was applied on the combination of the metabolomic abundance matrix (pathway level) and the microbiome relative abundance matrix (genera level) after fine-tuning the numbers of dimensions and variables to select using a k-fold cross-validation procedure. The samples and the selected variables were then clustered using k-means bi-clustering. The optimal number of sample clusters was defined using the gap statistic. When relevant, comparisons were performed using non-parametric Wilcoxon-Mann-Whitney rank sum test and a p-value threshold cutoff at 0.05 was considered. Correlation were evaluated using Pearson's correlation together with Pearson's correlation test.

It will be understood that, while various aspects of the present disclosure have been illustrated and described by way of example, the invention claimed herein is not limited thereto, but may be otherwise variously embodied according to the scope of the claims presented in this and/or any derivative patent application.

### REFERENCES

1. Dominguez-Bello, M. G. et al. Partial restoration of the microbiota of cesarean-born infants via vaginal microbial transfer. Nat. Med. 22, 250-253 (2016).
2. Dominguez-Bello, M. G. et al. Delivery mode shapes the acquisition and structure of the initial microbiota across multiple body habitats in newborns. Proc. Natl. Acad. Sci. U.S.A. 107, 11971-11975 (2010).
3. Ward, T. L. et al. Development of the Human Mycobiome over the First Month of Life and across Body Sites. mSystems 3, (2018).
4. Chu, D. M. et al. Maturation of the infant microbiome community structure and function across multiple body sites and in relation to mode of delivery. Nat. Med. 23, 314-326 (2017).
5. Bouslimani, A. et al. Molecular cartography of the human skin surface in 3D. Proc. Natl. Acad. Sci. U.S.A. 112, E2120-E2129 (2015).
6. Grice, E. A. et al. Topographical and temporal diversity of the human skin microbiome. Science (80-.). 324, 1190-1192 (2009).
7. Mukherjee, S. et al. Sebum and Hydration Levels in Specific Regions of Human Face Significantly Predict the Nature and Diversity of Facial Skin Microbiome. Sci. Rep. 6, (2016).
8. Oh, J., Byrd, A. L., Park, M., Kong, H. H. & Segre, J. A. Temporal Stability of the Human Skin Microbiome. Cell 165, 854-866 (2016).
9. Naik, S. et al. Compartmentalized control of skin immunity by resident commensals. Science (80-.). 337, 1115-1119 (2012).
10. Nakatsuji, T. et al. Antimicrobials from human skin commensal bacteria protect against Staphylococcus aureus and are deficient in atopic dermatitis. Sci. Transl. Med. 9, (2017).
11. Erin Chen, Y., Fischbach, M. A. & Belkaid, Y. Skin microbiota-host interactions. Nature 553, 427-436 (2018).
12. Barnard, E., Shi, B., Kang, D., Craft, N. & Li, H. The balance of metagenomic elements shapes the skin microbiome in acne and health. Sci. Rep. 6, 39491 (2016).
13. Fitz-Gibbon, S. et al. Propionibacterium acnes strain populations in the human skin microbiome associated with acne. J. Invest. Dermatol. 133, 2152-2160 (2013).
14. Agak, G. W. et al. Propionibacterium acnes induces an IL-17 response in acne vulgaris that is regulated by vitamin A and vitamin D. J. Invest. Dermatol. 134, 366-373 (2014).
15. Gao, Z., Tseng, C. H., Strober, B. E., Pei, Z. & Blaser, M. J. Substantial alterations of the cutaneous bacterial biota in psoriatic lesions. PLoS One 3, (2008).
16. Van Rensburg, J. J. et al. The human skin microbiome associates with the outcome of and is influenced by bacterial infection. MBio 6, (2015).
17. Byrd, A. L. et al. Staphylococcus aureus and Staphylococcus epidermidis strain diversity underlying pediatric atopic dermatitis. Sci. Transl. Med. 9, (2017).
18. Kong, H. H. & Segre, J. A. Skin microbiome: Looking back to move forward. Journal of Investigative Dermatology 132, 933-939 (2012).
19. Roth, R. R. & James, W. D. Microbial Ecology of the Skin. Annu. Rev. Microbiol. 42, 441-464 (1988).
20. Schmidt, T. S. B., Raes, J. & Bork, P. The Human Gut Microbiome: From Association to Modulation. Cell 172, 1198-1215 (2018).
21. Shaffer, M., Armstrong, A. J. S., Phelan, V. V., Reisdorph, N. & Lozupone, C. A. Microbiome and metabolome data integration provides insight into health and disease. Translational Research 189, 51-64 (2017).
22. Maruvada, P., Leone, V., Kaplan, L. M. & Chang, E. B. The Human Microbiome and Obesity: Moving beyond Associations. Cell Host and Microbe 22, 589-599 (2017).
23. Valles-Colomer, M. et al. The neuroactive potential of the human gut microbiota in quality of life and depression. Nat. Microbiol. 4, 623-632 (2019).
24. Sharon, G. et al. Human Gut Microbiota from Autism Spectrum Disorder Promote Behavioral Symptoms in Mice. Cell 177, 1600-1618.e17 (2019).
25. Lavelle, A. & Sokol, H. Gut microbiota-derived metabolites as key actors in inflammatory bowel disease. Nature Reviews Gastroenterology and Hepatology 17, 223-237 (2020).
26. Liu, Y. et al. Gut Microbiome Fermentation Determines the Efficacy of Exercise for Diabetes Prevention. Cell Metab. 31, 77-91.e5 (2020).
27. Hertel, J. et al. Integrated Analyses of Microbiome and Longitudinal Metabolome Data Reveal Microbial-Host Interactions on Sulfur Metabolism in Parkinson's Disease. Cell Rep. 29, 1767-1777.e8 (2019).
28. Vojinovic, D. et al. Relationship between gut microbiota and circulating metabolites in population-based cohorts. Nat. Commun. 10, 1-7 (2019).
29. Liu, H. et al. Alterations in the gut microbiome and metabolism with coronary artery disease severity. Microbiome 7, 68 (2019).
30. Chen, M. X., Wang, S. Y., Kuo, C. H. & Tsai, I. L. Metabolome analysis for investigating host-gut microbiota interactions. Journal of the Formosan Medical Association 118, S10-S22 (2019).
31. Hardman, M. J., Moore, L., Ferguson, M. W. J. & Byrne, C. Barrier formation in the human fetus is patterned. J. Invest. Dermatol. 113, 1106-1113 (1999).
32. Hardman, M. J. Patterned acquisition of skin barrier function during development. Development 125, 1541-1552. (1998).
33. Stamatas, G. N., Nikolovski, J., Luedtke, M. A., Kollias, N. & Wiegand, B. C. Infant Skin Microstructure Assessed In Vivo Differs from Adult Skin in Organization and at the Cellular Level. Pediatr. Dermatol. 27, 125-131 (2010).
34. Nikolovski, J., Stamatas, G. N., Kollias, N. & Wiegand, B. C. Barrier function and water-holding and transport properties of infant stratum corneum are different from adult and continue to develop through the first year of life. J. Invest. Dermatol. 128, 1728-1736 (2008).
35. Mack, M. C. et al. Water-Holding and Transport Properties of Skin Stratum Corneum of Infants and Toddlers Are Different from Those of Adults: Studies in Three Geographical Regions and Four Ethnic Groups. Pediatr. Dermatol. 33, 275-282 (2016).
36. Walters, R. M., Khanna, P., Chu, M. & Mack, M. C. Developmental Changes in Skin Barrier and Structure during the First 5 Years of Life. Skin Pharmacol. Physiol. 29, 111-118 (2016).
37. Visscher, M. O., Chatterjee, R., Munson, K. A., Pickens, W. L. & Hoath, S. B. Changes in diapered and nondiapered infant skin over the first month of life. Pediatr. Dermatol. 17, 45-51 (2000).
38. Yosipovitch, G., Maayan-Metzger, A., Merlob, P. & Sirota, L. Skin barrier properties in different body areas in neonates. Pediatrics 106, 105-108 (2000).
39. Saijo, S. & Tagami, H. Dry Skin of Newborn Infants: Functional Analysis of the Stratum Corneum. Pediatr. Dermatol. 8, 155-159 (1991).
40. Hoeger, P. H. & Enzmann, C. C. Skin physiology of the neonate and young infant: A prospective study of functional skin parameters during early infancy. Pediatr. Dermatol. 19, 256-262 (2002).
41. Iinuma, K. et al. Involvement of Propionibacterium acnes in the augmentation of lipogenesis in hamster sebaceous glands in vivo and in vitro. J. Invest. Dermatol. 129, 2113-2119 (2009).
42. van Smeden, J., Janssens, M., Goons, G. S. & Bouwstra, J. A. The important role of stratum corneum lipids for the cutaneous barrier function. Biochim. Biophys. Acta—Mol. Cell Biol. Lipids 1841, 295-313 (2014).
43. Kaiser, J. C. et al. Repression of branched-chain amino acid synthesis in Staphylococcus aureus is mediated by isoleucine via CodY, and by a leucine-rich attenuator peptide. PLoS Genet. 14, e1007159 (2018).
44. Phillips, C. D. et al. Microbiome structural and functional interactions across host dietary niche space. in Integrative and Comparative Biology 57, 743-755 (Oxford University Press, 2017).
45. Callahan, B. J. et al. DADA2: High-resolution sample inference from Illumina amplicon data. Nat. Methods 13, 581-583 (2016).
46. Segota, I. & Long, T. A high-resolution pipeline for 16S-sequencing identifies bacterial strains in human microbiome. bioRxiv 565572 (2019). doi:10.1101/565572
47. McMurdie, P. J. & Holmes, S. phyloseq: An R Package for Reproducible Interactive Analysis and Graphics of Microbiome Census Data. PLoS One 8, e61217 (2013).
48. Evans, A. M., DeHaven, C. D., Barrett, T., Mitchell, M. & Milgram, E. Integrated, nontargeted ultrahigh performance liquid chromatography/electrospray ionization tandem mass spectrometry platform for the identification and relative quantification of the small-molecule complement of biological systems. Anal. Chem. 81, 6656-6667 (2009).
49. Rohart, F., Gautier, B., Singh, A. & Lê Cao, K.-A. mixOmics: An R package for ‘omics feature selection and multiple data integration. PLOS Comput. Biol. 13, e1005752 (2017).
50. Lê, S., Josse, J. & Husson, F. FactoMineR: An R package for multivariate analysis. 25, 1-18 (2008).

1. A method of evaluating skin health, comprising:
observing microbiome and metabolome clusters on a surface area of said skin; and
assessing said skin health based on the make-up of said microbiome and metabolome clusters.

2. The method of claim 1:
wherein an abundance of Cutibacterium sp. in said microbiome is an indication of a ceramide- and lipid-rich, relatively dryer and more basic environment.

3. The method of claim 1:
wherein an abundance of Staphylococcus sp. in said microbiome is an indication of a lysine- and sugar-rich, more hydrated and acidic environment.

4. The method of claim 1,
wherein an abundance of Streptococcus sp. in said microbiome is independent of the presence of any particular metabolomic profile.

5. Use of the method of claim 1 to deduce treatment benefits on skin traits, including but not limited to skin moisturization and skin barrier function.
